(19) United States
(12) Patent Application Publication
(10) Pub. No.: US 2021/0335458 A1
(43) Pub. Date: Oct. 28, 2021

(54) SELF-REPORTING SYSTEM AND METHODS FOR HEALTH SCREENING

(71) Applicant: NineSquared81, LLC, Grand Haven, MI (US)

(72) Inventor: Paul McMullen, Grand Haven, MI (US)

(21) Appl. No.: 17/238,789

(22) Filed: Apr. 23, 2021

Related U.S. Application Data
(60) Provisional application No. 63/014,254, filed on Apr. 23, 2020.

(51) Int. Cl. G16H 10/20; G16H 10/65; G16H 40/67; G07C 9/25; G06Q 50/26; G06K 7/14; G06K 7/10; G06K 19/06; G06Q 10/10; G16H 50/80; G06Q 30/00; G06Q 50/20

(52) U.S. Cl. CPC G16H 10/20; G16H 10/40; G16H 40/67; G07C 9/253; G06Q 50/265; G06K 7/1417; G06K 7/1095; G06K 19/06037; G06K 19/06112; G06Q 10/103; G06Q 10/105; G16H 50/80; G06Q 30/018; G06Q 50/205; G16H 10/65

(57) ABSTRACT

A system and methods are disclosed for health screening users to help reduce the spread of a contagious disease. In one embodiment, an employee of an organization uses a mobile device, prior to arriving at a facility of the organization (e.g., a building, etc.), to take a “selfie” photo while their temperature is being taken. This might be done, for example, when the employee is at home. The employee enters the temperature reading into an application running on the mobile device and the application transmits the photo and temperature reading to the employee's manager for approval to work. Embodiments of the present disclosure enable health screening of users prior to their arrival at a facility, thereby potentially avoiding delays at the facility entrance that may cause long lines, as well as potentially reducing the number of staff required to perform the screening at the entrance.

### STATEMENT OF RELATED APPLICATIONS

The present application claims priority to, and incorporates fully by reference, U.S. Provisional Patent Application No. 63/014,254 filed Apr. 23, 2020.

### FIELD OF THE INVENTION

The present disclosure relates to health screening.

### BACKGROUND

Health screening has become more common in organizations (e.g., private companies, government agencies, etc.) as a means of containing the spread of contagious diseases such as COVID-19.

### BRIEF DESCRIPTION OF THE DRAWINGS

FIG. 1 depicts a flow diagram of aspects of a first method for health screening, in accordance with one embodiment of the present disclosure.

FIG. 2 depicts a flow diagram of aspects of a second method for health screening, in accordance with one embodiment of the present disclosure.

FIG. 3 depicts a block diagram of an illustrative computer system operating in accordance with aspects and implementations of the present disclosure.

### DETAILED DESCRIPTION

Some facilities (e.g., a commercial building, a school building, etc.) require checking whether a person might be ill prior to allowing the person to enter the facility. This has become more common since the arrival of COVID-19. Such checking might include a body temperature reading, a verbal or printed questionnaire (e.g., including a question such as “Have you traveled outside the United States in the last 30 days, etc.), and so forth.

Embodiments of the present disclosure enable health screening of users prior to their arrival at a facility, thereby potentially avoiding delays at the facility entrance (which may cause long lines), as well as potentially reducing the number of staff required to perform the screening at the entrance.

In accordance with one embodiment of the present disclosure, an employee of an organization uses a mobile device (e.g., a smartphone, a tablet, etc.) to take a “selfie” photo while their temperature is being taken. The employee enters the temperature reading into an application running on their mobile device (henceforth referred to as “the app”), and the app transmits the photo and temperature reading to the employee's manager for approval to work. In one embodiment, prior to transmitting the photo and temperature reading, the application first checks the time/date stamp of the photo, which may be, for example, embedded in the photo file, or stored in metadata external to the file, etc. In one example, the application will not accept a photo that is older than a chosen threshold (e.g., one hour, 12 hours, etc.).

When the supervisor approves the request, the employee's mobile device receives a unique QR code and the app displays the QR code on a green screen (i.e., the QR code is displayed on a green background), indicating that the employee “passed” (i.e., they have been granted permission to report to work). Upon the employee's arrival at a facility of the employer (e.g., a building, a campus, etc.), the QR code is scanned at the entrance, and the combination of the QR code and green screen allows the employee to enter the facility. In one embodiment, the QR code may be matched against a copy of the QR code maintained by the app executing on the supervisor's mobile device.

In one embodiment, the QR code has a 12-hour window for access. The green screen persists for 12 hours, after which the screen turns yellow and displays a countdown (e.g., an additional hour, etc.), after which the yellow screen turns to red and the employee's manager is notified that the employee's access has ended.

The above process can be repeated for each “shift”. It should be noted that the 12-hour window is merely exemplary, and in other embodiments the window might have a different duration (e.g., 8 hours, etc.). Similarly, the two-hour-before-expiration timing of the yellow screen could differ in other embodiments (e.g., it could occur one hour from expiration, etc.). Similarly, some other embodiments might employ colors and/or color sequences other than green-yellow-red. Moreover, it should be noted that while embodiments of the present disclosure employ a QR code, some other embodiments might employ some other type of image-based code (e.g., a traditional barcode, etc.).

In accordance with one embodiment, the application provides 24-hour green after one or both of the following have occurred: (1) the employee has received the appropriate vaccine(s)/booster(s); (2) the employee has received positive antibody test results.

In one embodiment, the app stores medical information associated with the employee on the mobile device. This information may also be backed up to the employee's cloud drive, and may be shared with workplace human resources or other authorities having jurisdiction.

It should be noted that while the term “manager” is used in the present disclosure, in some organizations an employee's superior might have some other title (e.g., “group leader”, “supervisor”, etc.).

It should further be noted that the system and methods of the present disclosure may be adapted to other domains, such as health screening for mass transit, health screening for school (in which case, users comprise students, rather than employees), and so forth.

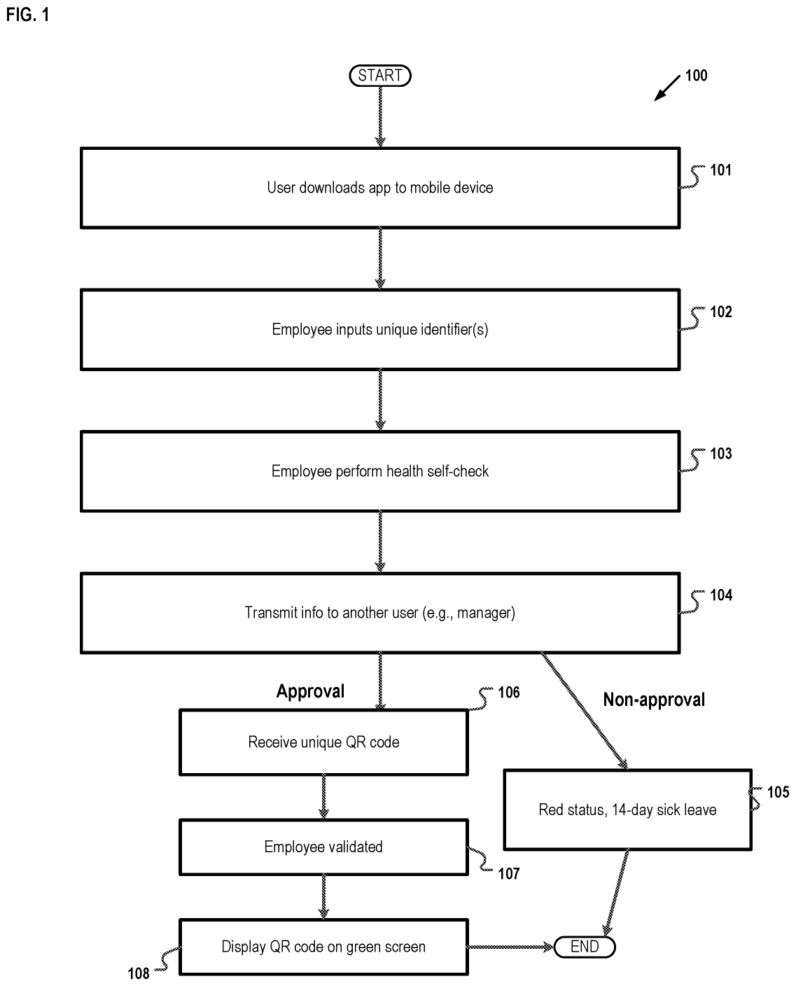

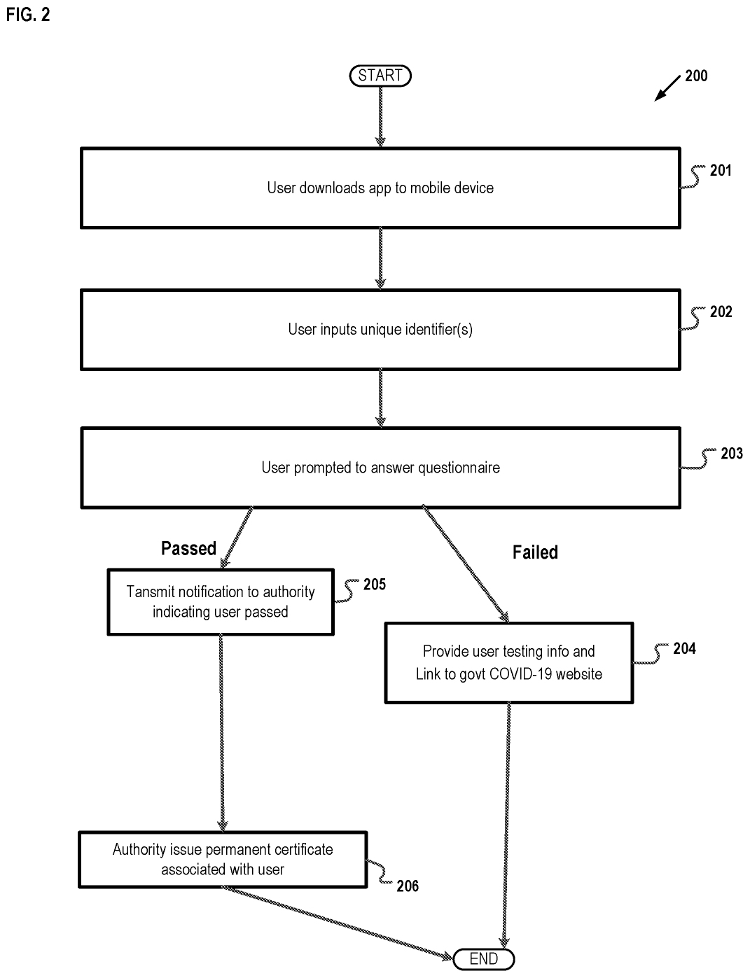

FIG. 1 depicts a flow diagram of aspects of a method 100 for health screening, in accordance with one embodiment of the present disclosure. The method, as well as the method of FIG. 2 below, may be used to help reduce the spread of a contagious disease (e.g., COVID-19, tuberculosis, etc.).

Method 100 may be performed by processing logic that may comprise hardware (circuitry, dedicated logic, etc.), software (such as is run on a general-purpose computer system or a dedicated machine), or a combination of both. In some embodiments, blocks depicted in FIG. 1 might be performed concurrently, or in a different order than that depicted.

At block 101, a user downloads and installs an app on his or her mobile device (e.g., a smartphone, etc.). In one embodiment, the app will be sent from the employer to register a worker and link them to their managers and/or substitutes with unique Username/Password/employee ID. In one embodiment, the user may indicate that he or she is an employee of the organization (e.g., by choosing an “employee profile” from a plurality of profiles during setup of the app, when the app is run for the first time; etc.).

It should be noted that in some examples, the user might not yet be an employee at the time that the app is downloaded, but is later hired as an employee after the download, but prior to the execution of block 102 below. As one example, the user might be applying for a job at the organization, and might be reporting for an in-person interview. As another example, the user might currently be a contract employee for the organization, rather than an employee of the organization. For the remainder of the method, the user will be referred to as an employee.

At block 102, the employee inputs one or more unique identifiers into the app (e.g., an employee number, etc.) and agrees to terms and conditions.

At block 103, the employee performs a self-health check before arriving at the organization's facility (e.g., at home, etc.). In one embodiment, the self-health check comprises the following actions, performed through the app: taking a “selfie” photo while their temperature is being taken with a thermometer, such that the temperature reading is visible in the photo; entering the temperature reading; answering a symptom survey; declaring that (1) the temperature reading is authentic and (2) they understand that if they lie or withhold information, they may be terminated (e.g., via one or more checkboxes, etc.); waiving HIPAA (Health Insurance Portability and Accountability Act) privacy; and submitting the information (e.g., hitting a “submit” button that is displayed in the app, etc.).

In one embodiment, the application checks the time/date stamp of the photo, which may be, for example, embedded in the photo file, or stored in metadata external to the file, etc. In one example, the application will not accept a photo that is older than a chosen threshold (e.g., one hour, 12 hours, etc.).

It should be noted that in some embodiments, the photo may be taken by another person, rather than being a selfie taken by the employee. In addition, the photo might be taken outside the app, rather than from within the app.

Similarly, in some embodiments, the reporting of the temperature reading might be part of the symptom survey, rather than a separate step. In addition, in some embodiments the employee may also verify their identity through the app before submitting the information (e.g., via fingerprint recognition, facial recognition, etc.). It should be noted that in some embodiments, a Bluetooth FDA-approved thermometer might be used to take the temperature reading and transmit it to the app, thereby obviating the need for the user to manually provide the temperature reading to the app. It should further be noted that in some embodiments, additional vital signs data (e.g., heart rate, etc.) may be provided to the app. In some such embodiments, a wearable worn by the user, or the user's mobile device, might provide the body temperature and/or some or all of the vital sign data.

If their temp is high then the employee must submit 14 successive normal temp photos 24-hrs apart to be allowed to come back to work. The medical definition of a low-grade fever is 100.4F, but 99.7F is being published as an elevated body temperature in COVID-19 cases with the symptom of chills.

In one embodiment, the self-health check at block 103 might be performed in response to an incoming message. In some implementations, the app might receive an invitation to report from a manager (e.g., from the manager's mobile device running the app, etc.). In some such implementations, the invitation might be transmitted from the manager's mobile device (e.g., through the app, etc.), while in some other implementations the invitation might be transmitted from another device and/or from another application. It should be noted that in some examples the invitation might be sent in an automated fashion (e.g., based on a schedule, etc.) rather than by the manager actively inviting the employee.

In some embodiments, the manager may have previously indicated to the app (e.g., during setup, etc.) that he or she is a manager of the organization by choosing a “manager profile” from the plurality of profiles, similar to an employee choosing the “employee profile,” as is disclosed in block 101 above. This indication may also involve, as in the case of an employee, inputting one or more unique identifiers into the app (e.g., an employee number, etc.) and agreeing to terms and conditions.

In some examples, the manager may also validate his or her own health to a supervisor (e.g., by performing a self-health check similar to the employee self-health check of block 103, etc.). In some such examples, the app may transmit the information to the supervisor for his or her approval, after which a subsequent chain of approval request(s) may occur (for example, a director approving or not approving the supervisor; if the supervisor is approved, a vice president approving or not approving the director, and so forth.). If the manager is not approved at a particular point in the chain, then execution of the method proceeds to block 105. It should be noted that in some implementations, each person in the chain might indicate an approval or non-approval through the app (e.g., running on their personal mobile device, etc.), while in some other implementations the approvals/non-approvals might occur outside the app, and then reported to the app.

At block 104, the information submitted at block 103 is transmitted to a computer system associated with another user (e.g., a mobile device that is associated with another employee of the organization and runs the app, etc.), for this user to review and approve/disapprove. In one example, the other user is a shift manager at the organization who is a different person than the employee's manager, while in some other examples, the other user is a manager to which the employee reports who is independent of shifts. For the remainder of the method, this user will be referred to as a manager.

In one embodiment, approval requires that the reported body temperature be below a threshold, and that the person in the photo is recognized as the employee in question. In some examples this recognition may be by the manager, while in some other examples the recognition may be automated in some fashion (for example, using a facial recognition algorithm that is capable of determining whether two facial photos are of the same person, where the second photo is a prior photo on file). As will be appreciated by those skilled in the art, some facial recognition algorithms work by computing a degree of match between two photos of faces, and comparing the degree of match to a threshold.

Upon approval, a message is transmitted to the employee's mobile device indicating that the employee has been approved to report to the organization's facility, and execution of the method proceeds to block 106. Upon non-approval (i.e., explicit disapproval or a lack of approval), execution of the method proceeds to block 105.

At block 105, which is reached as a result of non-approval (e.g., by the manager at block 104, by a supervisor or director or vice president prior to the transmittal of an invitation to the employee, etc.), the employee is placed on 14-day sick leave and the employee's app status is set to red. It should be noted that in some embodiments, block 105 might first check that approval was denied for medical reasons (e.g., based on temperature and/or reported symptoms, etc.), and not for some other reason, before placing the employee on 14-day sick leave.

At block 106, which is reached after the employee's mobile device receives the message indicating that the employee has been approved to report to the organization's facility, the app running on the employee's mobile device receives a unique QR code. In some implementations the QR code might be embedded in the message, while in some other implementations, it might be received from some other machine (e.g., a server, etc.). The employee can then report to the manager at the organization's facility. When the employee has reported, execution continues at block 107.

At block 107, the manager validates the employee via the app running on the manager's mobile device, and the manager's mobile device uses its camera to read the QR code on the green screen of the employee's mobile device. The manager's mobile device then sends to the employee's mobile device a message indicating that the employee has been validated. In one embodiment, the manager's mobile device also transmits the employee's health status to a supervisor. After block 107, execution proceeds to block 108.

At block 108, in response to the employee's mobile device receiving the message, the app running on the employee's mobile device displays the QR code on a green screen. The current time is used as a starting point for a particular length of time during which the screen is green (e.g., 12 hours, etc.), followed by a length of time during which the screen is yellow, followed by a red screen. When the red screen is reached, the employee's mobile device transmits a message to the manager's mobile device indicating that the access period for the employee has expired and the employee's work day is complete. In one embodiment, employee access at the facility may be controlled by one or more of a card-access system, a time-keeping system, the app on the manager's mobile device, etc., based on the state of the employee's app. After block 108, method 100 terminates.

FIG. 2 depicts a flow diagram of aspects of a second method 200 for health screening, in accordance with one embodiment of the present disclosure. Method 200 may be performed by processing logic that may comprise hardware (circuitry, dedicated logic, etc.), software (such as is run on a general-purpose computer system or a dedicated machine), or a combination of both. In some embodiments, blocks depicted in FIG. 2 might be performed concurrently, or in a different order than that depicted.

In contrast to method 100, which performs health screening for an employee of an organization, method 200 may be applicable to any person. Accordingly, method 200 refers to the person in question as the “user” of his or her mobile device.

It should be noted that, while method 200 is disclosed with respect to COVID-19, the method may be applicable to other contagious diseases. It should further be noted that in some embodiments, method 200 may be executed by the same app as method 100, while in other embodiments method 200 may be executed by a different app. In view of this, when method 200 refers to “app”, it is understood that this might be the same app referenced in method 100, or it might be a different app.

At block 201, a user downloads and installs an app on his or her mobile device. At block 202, the user inputs one or more unique identifiers into the app (e.g., his or her social security number, a license plate number, etc.) and agrees to terms and conditions.

At block 203, the user is prompted to answer a questionnaire comprising one or more COVID-19-related questions. In one embodiment, the questions include asking the user one or more of the following: (1) have you tested positive for COVID-19?; (2) if so, (a) what was the date of the test, and (b) have you completed the quarantine period?; (3) have you received results from an antibody test; (4) if so, have you been determined immune?; and (5) have you received a COVID-19 vaccination?

In one implementation, after the user has answered the questions, the app compares the answers to a set of one or more rules indicating whether the user has “PASSED” or “FAILED.” For example, there might be rules such as “IF (A4=YES) OR (A5=YES) THEN “PASSED.” If the user fails the questionnaire, the method continues to block 204, otherwise the method continues to block 205. If the user has passed the questionnaire, in accordance with one embodiment the user may be asked to provide one or more documents verifying his or her answers to the questionnaire (e.g., via photos that are provided to the app, via an upload to a cloud server, etc.). In some implementations, the user may also have to agree to a HIPAA waiver, and/or acknowledge that the document(s) are authentic to the best of their knowledge. In the latter case, the acknowledgement may also indicate that user is subject to prosecution if the documents are not authentic.

At block 204, the app provides the user with one or both of (a) information about where the user can go to get tested, and (2) a link to a government COVID-19 website. After block 204 has been completed, method 200 terminates.

At block 205, the app sends a notification to an authority that has jurisdiction to issue a “permanent certificate” (e.g., a government agency, a school administration, etc.), where the notification indicates that the user has passed the questionnaire. In one embodiment, the notification is sent via a message (e.g., an email, etc.), optionally providing access to the user's document(s) (e.g., as email attachment(s), as a link to file(s) on a server, etc.).

At block 206, the authority issues a “permanent certificate” associated with the user. In one embodiment, the certificate is transmitted to the app running on the user's mobile device, and in response, the app displays a screen that indicates that the user is “COVID-19 free.”

As will be appreciated by those skilled in the art, in some embodiments the system and methods of the present disclosure may be augmented with additional functionality, such as GPS tracking, providing daily check-ins, additional measurement criterion (e.g., symptom surveys, etc.) to track safety and efficacy.

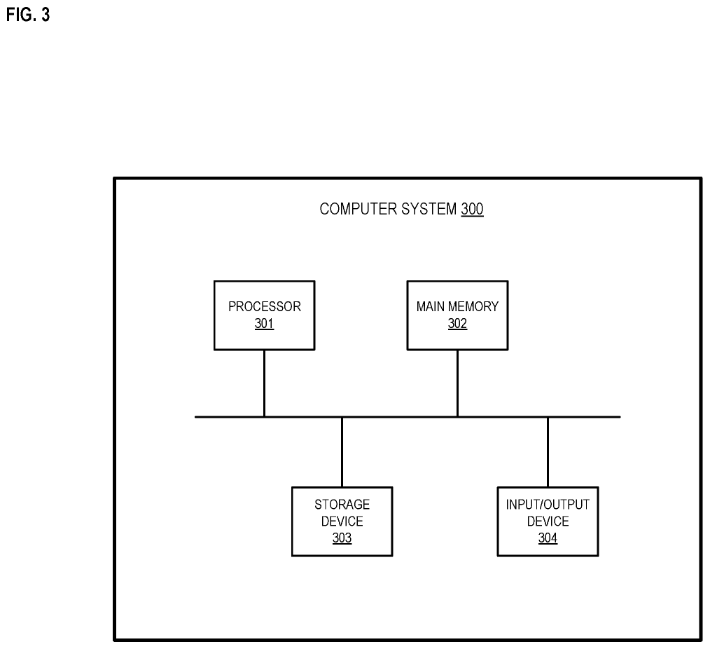

FIG. 3 depicts a block diagram of an illustrative computer system 300 operating in accordance with aspects and implementations of the present disclosure. Computer system 300 may be a personal computer (PC), a laptop computer, a tablet computer, a smartphone, or any other computing or communication device. As shown in FIG. 3, computer system 300 comprises processor 301, main memory 302, storage device 303, and input/output (I/O) device 304, interconnected as shown (e.g., via one or more busses, etc.)

Processor 301 represents one or more general-purpose processing devices such as a microprocessor, central processing unit, or the like. More particularly, processor 301 may be a complex instruction set computing (CISC) microprocessor, reduced instruction set computing (RISC) microprocessor, very long instruction word (VLIW) microprocessor, or a processor implementing other instruction sets or processors implementing a combination of instruction sets. Processor 301 may also be one or more special-purpose processing devices such as an application specific integrated circuit (ASIC), a field programmable gate array (FPGA), a digital signal processor (DSP), network processor, or the like. Processor 301 is capable of executing instructions stored in main memory 302 and storage device 303, including instructions corresponding to the methods of FIGS. 1 and 2 above; of reading data from and writing data into main memory 302 and storage device 303; and of receiving input signals and transmitting output signals to input/output device 304. While a single processor is depicted in FIG. 3 for simplicity, computer system 300 might comprise a plurality of processors.

Main memory 302 is capable of storing executable instructions and data, including instructions and data corresponding to the methods of FIGS. 1 and 2 above, and may include volatile memory devices (e.g., random access memory [RAM]), non-volatile memory devices (e.g., flash memory), and/or other types of memory devices.

Storage device 303 is capable of persistent storage of executable instructions and data, including instructions and data corresponding to the method of FIG. 1 above, and may include a magnetic hard disk, a Universal Serial Bus [USB] solid state drive, a Redundant Array of Independent Disks [RAID] system, a network attached storage [NAS] array, etc. While a single storage device is depicted in FIG. 3 for simplicity, computer system 300 might comprise a plurality of storage devices.

I/O device 304 receives input signals from a user of computer system 300, forwards corresponding signals to processor 301, receives signals from processor 301, and emits corresponding output signals that can be sensed by the user. The input mechanism of I/O device 304 might be an alphanumeric input device (e.g., a keyboard, etc.), a touchscreen, a cursor control device (e.g., a mouse, a trackball, etc.), a microphone, etc., and the output mechanism of I/O device 304 might be a liquid-crystal display (LCD), a cathode ray tube (CRT), a speaker, etc. While a single I/O device is depicted in FIG. 3 for simplicity, computer system 300 might comprise a plurality of I/O devices.

It is to be understood that the above-described embodiments are merely illustrative, and that many variations of the above-described embodiments can be devised by those skilled in the art without departing from the scope of this disclosure. It is therefore intended that such variations be included within the scope of the following claims and their equivalents.

What is claimed:

1. A method for health screening employees of an organization to help reduce the spread of a contagious disease, the method comprising:
executing, by a first mobile device associated with a first employee of the organization, an application for health screening;
obtaining, by the application executing on the first mobile device, a photo of the first employee, wherein the photo shows the first employee's face and a thermometer taking the first employee's temperature, and wherein a temperature reading of the thermometer is visible in the photo;
providing the photo to a second mobile device associated with a second employee of the organization for approval, wherein approval is based on the temperature reading and a recognition that the photo is of the employee; and
when, and only when, approval is obtained based on the temperature reading and the recognition:
(i) receiving, by the application executing on the first mobile device, a unique image-based code;
(ii) displaying, by the application executing on the first mobile device, the unique image-based code on a green background;
(iii) in response to
(a) the first employee arriving at an entrance of a facility associated with the organization, and
(b) a camera of the second mobile device reading the image-based code on the green background displayed by the first mobile device,
permitting the first employee to enter the facility.

2. The method of claim 1 wherein the photo is a selfie.

3. The method of claim 1 wherein the second employee is a superior of the first employee in the organization.

4. The method of claim 1 wherein the green background changes to another color in response to a difference between the current time and the time that the first employee entered the facility.

5. A method for health screening to help reduce the spread of a contagious disease, the method comprising:
executing, by a mobile device associated with a user, an application for health screening;
prompting, by the application, the user to answer a questionnaire comprising one or more COVID-19-related questions;
obtaining from the user, by the application, one or more documents verifying answers by the user to the questionnaire;
transmitting, by the application, a notification to an authority that has jurisdiction to issue a permanent certificate, wherein the notification indicates that the user has passed the questionnaire and provides access to the one or more documents;
receiving by the application, from the authority, an issued permanent certificate that is associated with the user; and
in response to receiving the issued permanent certificate, displaying on the mobile device, by the app, a screen that indicates that the user is COVID-19 free.

6. The method of claim 5 wherein the one or more documents are obtained by the app via one or more photos.

7. The method of claim 5 wherein the authority is a government agency.

8. The method of claim 5 wherein the authority is a school administration.
